(19) United States
(12) Patent Application Publication
(10) Pub. No.: US 2022/0257971 A1
(43) Pub. Date: Aug. 18, 2022

(54) FACIAL BEAUTY DEVICE AND MASK SHEET USED THEREFOR

(71) Applicant: LG HOUSEHOLD & HEALTH CARE LTD., Seoul (KR)

(72) Inventors: Eun Mi KIM, Seoul (KR); Kyung Sik JANG, Seoul (KR); Bok Hyun PACK, Seoul (KR)

(73) Assignee: LG HOUSEHOLD & HEALTH CARE LTD., Seoul (KR)

(21) Appl. No.: 17/611,519

(22) PCT Filed: May 15, 2020
(86) PCT No.: PCT/KR2020/006414

§ 371 Date: Nov. 15, 2021

(30) Foreign Application Priority Data
May 16, 2019 (KR) 10-2019-0057274

(51) Int. Cl. A61N 5/06

(52) U.S. Cl. CPC A61N 5/0616; A61N 2005/0632; A61N 2005/0626; A61N 2005/0665; A61N 2005/0647

(57) ABSTRACT

A facial beauty device according to an embodiment may comprise: a face body; a seating member coupled to the face body; and a support part disposed on one side of the face body. The face body may comprise: a first face body; a second face body; and a circuit board disposed between the first face body and the second face body. The circuit board may include a predetermined stimulation providing part disposed thereon. The second face body may include a mask sheet disposed at the inner side thereof.

### TECHNICAL FIELD

Embodiments relate to a facial beauty device, and more particularly, to a facial beauty device capable of intensive care of a face as a whole, and a mask sheet used therein.

### BACKGROUND ART

With the development of beauty industry, people's interest is increasing in skin care services for improving skin tone or wrinkles as well as products such as basic cosmetics and color cosmetics.

Meanwhile, the effect of skin care generally appears when the skin care is received continuously for a long period of time rather than when the skin care is received for a short period of time. Therefore, it is important to receive continuous skin care in order for a user to acquire effects such as skin tone improvement or wrinkle improvement.

However, users have difficulty in continuing to receive skin care due to reasons such as lack of access to places where skin care services are provided and lack of time.

Therefore, it is required to develop a facial beauty device that provides a skin care function at a place desired by the user and at a time desired by the user.

Meanwhile, a skin care device in a related art provides a single function such as an LED skin regeneration function, but has not been able to provide a care effect customized to for each skin area of the face.

In addition, in the related art, it is difficult to uniformly irradiate LED light over the entire face, and accordingly, there is a problem that a uniform care effect cannot be obtained over the entire face.

Further, in the related art, when LED elements are densely disposed so as to correspond to the entire face, not only it is difficult to implement a flexible mask due to interference between the LED elements, but also there is a problem of a technical contradiction that overheating occurs.

In addition, in the related art, there is a problem that a uniform treatment or care is performed only by the functions set in the product itself, which does not consider the condition of the skin to be cared for by the user.

In particular, in the related art, it is not possible to recognize the issue of checking in real time how much skin elasticity is improved during skin care.

In addition, according to the related art, a mask for facial care is used spaced apart from the face, and even though the mask is to be brought into close contact with the face, it is difficult to use the mask in close contact with the face.

In addition, according to the related art, a light-emitting device and a vibration device for skin care are applied in a form of individual devices.

However, when the light-emitting device and the vibration device are disposed as separate devices, there is a technical contradiction that uniform care may not be implemented over the entire face because the light-emitting device may not be disposed in an area where the vibration device is disposed.

In addition, according to the related art, when an LED is used in a skin care device, even though a single wavelength LED is used or an LED of a plurality of various wavelengths is used, each individual LED emits light with a predetermined wavelength.

Accordingly, in the conventional LED mask, the number of LEDs that may be mounted is limited, and there is a limit that an emission wavelength may not be used in a variable type depending on the user's condition.

### DISCLOSURE

### Technical Problem

The embodiment is directed to providing a facial beauty device capable of customized multi-care for each skin area and realizing a three-dimensional flexible mask.

In addition, the embodiment is directed to providing a facial beauty device that may implement close contact with the face, may intensively care for the skin, and may be washed with water and waterproof.

In addition, the embodiment is directed to providing a facial beauty device that may protect a curved surface of the face while being in close contact with the curved surface of the face.

In addition, the embodiment is directed to providing a facial beauty device capable of increasing a skin efficacy and effect through a multi-use of a formulation and the beauty device, and a mask sheet used therefor.

In addition, the embodiment is directed to providing a facial beauty device in which a microcurrent electrode of a stimulation providing part may be energized to the face even after a mask sheet is attached, and the mask sheet used therefor.

In addition, the embodiment is directed to providing a facial beauty device that may check the use of the mask sheet and the progress of skin care time in real time, and a mask sheet used therefor.

In addition, the embodiment is directed to providing a facial beauty device that may increase the overall uniform face care effect.

In addition, the embodiment is directed to providing a facial beauty device that may increase the overall uniform face care effect by evenly irradiating LED light to the entire facial skin even though a light-emitting module is disposed in an appropriate range.

In addition, the embodiment is directed to providing a facial beauty device that may further improve an effect of increasing the dermal density of facial skin, increasing skin brightness, and increasing skin elasticity due to excellent penetration into the facial skin.

In addition, the embodiment is directed to providing a facial beauty device that may measure the skin elasticity in real time during skin care.

In addition, the embodiment is directed to providing a facial beauty device that may increase the effect of skin care by increasing the adhesion between a facial mask and the facial skin.

In addition, the embodiment is directed to providing a facial beauty device capable of simultaneously implementing light and ultrasound for face care.

In addition, the embodiment is directed to providing a facial beauty device that may measure the skin elasticity of a user before skin care, then select an appropriate LED wavelength according to the user, and then perform skin care by the corresponding LED wavelength.

In addition, the embodiment is directed to providing a facial beauty device that may maximize the effect of skin care by precisely measuring a thickness of the adipose tissue layer of a user's skin before skin care and performing skin care by controlling the optimal LED wavelength suitable for the corresponding user.

In addition, the embodiment is directed to providing a facial beauty device that may exert an intensive and optimal care effect in consideration of a skin thickness of a user and treatment or care depth after precisely measuring the skin thickness of the user.

### Technical Solution

A facial beauty device according to an embodiment may include a face body 310, a mounting member 320 coupled to the face body 310, and a support part 350 disposed at one side of the face body 310.

The face body 310 may include a first face body 311, a second face body 312, and a circuit board 315 disposed between the first face body 311 and the second face body 312.

The circuit board 315 may include a predetermined stimulation providing part disposed thereon.

The second face body 312 may include a mask sheet 360 disposed at the inner side thereof.

The mask sheet 360 may include a predetermined opening so that an electrode provided in the stimulation providing part may be energized to a user's face even after the mask sheet 360 is attached.

The mask sheet 360 may include a plurality of openings 360h, the mask sheet 360 may include a first opening 360h1 in an area corresponding to the user's eyes, a second opening 360h2 in an area corresponding to the user's mouth, and a third opening 360h3 at a position corresponding to the stimulation providing part 316.

A second diameter D2 of the third opening 360h3 of the mask sheet 360 may be larger than a first diameter D1 of the stimulation providing part 316.

A hardness of the first face body 311 and that of the second face body 312 may be different from each other.

The stimulation providing part 316 may include one or more among an ion module 316b, a vibration module, or a light-emitting module.

The mounting member 320 may include a first mounting member 321 coupled to a first region of the second face body 312 and a second mounting member 322 coupled to a second region of the second face body 312.

A first frame 371 may be further included on the second face body 312.

The first frame 371 may include a first-first frame 371a disposed at a position corresponding around the user's eye area and a first-third frame 371c disposed at a position corresponding around the user's forehead.

The first frame 371 may include a first-second frame 371b disposed between the first-first frame 371a and the first-third frame 371c.

A horizontal width of the first-first frame may be smaller than a vertical width of the first-second frame.

The stimulation providing part may include a first light-emitting module 316a disposed on the face body 310, and the embodiment may further include a first diffusion plate 317a disposed on the first light-emitting module 316a.

The stimulation providing part may include a second light-emitting module 316a2 disposed on the circuit board 315, and the embodiment may further include a second diffusion plate 317b disposed on a side surface of the second light-emitting module 316a2 and a reflective material coating layer 317c disposed above the second diffusion plate 317b.

The stimulation providing part may include the first light-emitting module 316a disposed on the circuit board 315, and the embodiment may further include the first diffusion plate 317a disposed on the first light-emitting module 316a, and a diffraction optical pattern 318 disposed between the diffusion plates 317a.

The stimulation providing part may include a piezoelectric sensor 410 disposed on the circuit board 315, and the piezoelectric sensor 410 may include a flexible substrate 410a, a piezoelectric thin film layer 410b on the flexible substrate 410a, an electrode 410c on the piezoelectric thin film layer 410b, and a protective layer 410d on the electrode 410c.

The embodiment may further include an adhesive layer 312bp at an edge of an inner side 312b of the second face body.

The stimulation providing part may include a contact time sensing element 420 disposed on the circuit board 315, wherein the contact time sensing element 420 may include a plurality of electrical contacts 422 and a plurality of ports 423, and it is possible to determine whether the formulation of the mask sheet 360 remains by detecting a short-circuit between the electrical contacts by a monitor module 421.

The stimulation providing part may include an optical vibration composite element 430 disposed on the circuit board 315, and the optical vibration composite element 430 may include a first electrode layer 431 disposed on the circuit board 315, a flexible light-emitting layer 432 disposed on the first electrode layer 431, a second electrode layer 433 disposed on the flexible light-emitting layer 432, a flexible ultrasonic layer 434 disposed on the second electrode layer 433, and a third electrode layer 435 disposed on the flexible ultrasonic layer 434.

The stimulation providing part may include a wavelength tunable light-emitting module 440 disposed on the circuit board 315, and the wavelength tunable light-emitting module 440 may include a piezoelectric substrate 441 disposed on the circuit board 315 and a wavelength tunable light-emitting device 442 disposed on the piezoelectric substrate 441.

The stimulation providing part may include a skin thickness measurement element 450 disposed on the circuit board 315, and the skin thickness measurement element 450 may include one or more ultrasonic transducers 453 disposed on the circuit board 315 and one or more RF electrodes 451 and 452 disposed on both sides of the ultrasonic transducer 453.

The stimulation providing part may include a linear ultrasound module 460 disposed on the circuit board 315, and the linear ultrasound module 460 may include a cylindrical conversion element 461 disposed on the circuit board 315 and an imaging element 464 disposed on the cylindrical conversion element 461.

The stimulation providing part may include a composite skin thickness measurement element 470 disposed on the circuit board 315, and the composite skin thickness measurement element 470 may include an ultrasound module 471 disposed on the circuit board 315 and a light detection module 472.

The mask sheet according to the embodiment may be used in a facial beauty device having the stimulation providing part on the face body 310 and may include an opening through which a predetermined electrode provided in the stimulation providing part of the facial beauty device may pass by providing a predetermined opening.

The opening may include the first opening 360h1 in the area corresponding to the user's eyes, the second opening 360h2 in the area corresponding to the user's mouth, and the third opening 360h3 at the position corresponding to the stimulation providing part 316.

The second diameter D2 of the third opening 360h3 may be larger than the first diameter D1 of the stimulation providing part 316.

### Advantageous Effects

According to the embodiment, it is possible to provide a three dimensional (3D) flexible mask capable of customized multi-care for each skin area.

For example, according to the embodiment, it is possible to provide an increase in skin elasticity and a skin regeneration effect in a wide area such as cheeks and a forehead, to provide a galvanic effect for wrinkle improvement around the eyes, and to perform customized multi-care for each part such as a close V-line lifting effect on chin skin.

In addition, according to the embodiment, there is an effect that it is possible to implement close contact with the face, to intensively care for the skin, and to wash with water and be waterproof by applying the flexible material such as silicone, TPE, or urethane to the face body, and at the same time, by the face lifting effect by the mounting member 320 described later.

For example, according to the embodiment, when the user uses the face body, the flexible material is not simply placed on the face, but the face body is lifted by pulling the mounting member which is a stretch bending part to the chin, and at the same time, is in close contact with the face, so that effects such as heat, galvanic, light, etc. may be further maximized as the face body is in direct close contact with the face.

In addition, the embodiment is provided with a frame on the face body, so that there is a technical effect that it is possible to prevent excessive pressing of the forehead, nose, and eye area having some curved surfaces of the face when the face body is in close contact with the face, and to protect the curved surface of the face while being in close contact with the curved surface of the face.

For example, according to an embodiment, there is a technical effect that it is possible to minimize the compression of a bone by a first-third frames 371c disposed around the forehead, and to protect important areas of the face such as eyes and forehead by a first-first frame 371a disposed around the eye area when a chin area of the flexible face body is in close contact with the face.

In addition, it is possible to enhance a protection function of the forehead area and the eye area while strengthening a frame structure by a first-second frame 371b disposed between the first-first frame 371a and the first-third frame 371c.

In addition, according to the embodiment, there is a technical effect capable of increasing the skin efficacy and effect through the multi-use of the formulation and the beauty device by wearing together with a mask sheet 360 containing the formulation.

In addition, in the embodiment, the mask sheet 360 has a predetermined opening so that the microcurrent electrode of the stimulation providing part may be energized to the face even after the mask sheet 360 is attached.

For example, in the embodiment, a second diameter D2 of a third opening 360h3 of the mask sheet 360 may be larger than a first diameter D1 of a galvanic module 316b, and accordingly. the embodiment may implement a mask sheet form in which a microcurrent of the galvanic module 316b may be directly energized to the skin.

In addition, in the embodiment, when the galvanic module 316b, which is an ion module, is provided on a circuit board 315, a predetermined opening (not shown) may also be provided in a second face body 312 so that the galvanic module 316b may be in direct contact with the skin.

In addition, according to a second embodiment, light emitted from a first light-emitting module 316a is diffused by including a first diffusion plate 317a disposed on the first light-emitting module 316a, so that there is a technical effect that it is possible to increase the overall uniform face care effect by evenly irradiating LED light to the entire facial skin even though the light-emitting module is disposed in an appropriate range.

In addition, according to a third embodiment, light emitted from a second light-emitting module 316a2 is diffused by a second diffusion plate 317b disposed on the side surface of the second light-emitting module 316a2 and the reflective material coating layer 317c, so that there is a technical effect that it is possible to increase the overall uniform face care effect by evenly irradiating LED light to the entire facial skin even though the light-emitting module is disposed in an appropriate range.

In addition, according to a fourth embodiment, an LED light source emitted from the first light-emitting module 316a may be uniformly concentrated and irradiated on the face by the diffraction optical pattern 318 disposed between or under the first diffusion plate 317a, and thus it is possible to improve an effect of increasing the dermal density of the facial skin, increasing skin brightness, and increasing skin elasticity due to excellent penetration into the facial skin.

In addition, a fifth embodiment includes a piezoelectric sensor 410 having a piezoelectric thin film layer 410b disposed on a flexible substrate 410a, and accordingly, there is a special technical effect that it is possible to measure skin elasticity in real time during skin care.

In addition, in a sixth embodiment, the adhesion to the skin is increased by disposing an adhesive layer 312bp made of an acrylic monomer material with high adhesion and biocompatibility at the edge of the second face body, and accordingly, the adhesion between the face mask and the facial skin is increased, thereby increasing the effecting of skin care.

In addition, in a seventh embodiment, there is a special technical effect that it is possible to check the degree of use of the mask sheet 360, the progress of skin care time, and the like in real time by accurately measuring the moisture content of the cosmetic formulation contained in the mask sheet 360 through a contact time sensing device 420.

In addition, according to an eighth embodiment, there is a technical effect that it is possible to simultaneously implement light and ultrasonic waves for facial care by providing an optical vibration composite element 430 including a flexible light-emitting layer 432 and a flexible ultrasonic layer 434.

In addition, according to the eighth embodiment, as light and ultrasonic waves may be simultaneously implemented by one optical vibration composite element 430, the optical vibration composite element 430 may be densely disposed over the entire face, and accordingly, there is a special technical effect that it is possible to implement very uniform care over the entire face.

In addition, according to a ninth embodiment, there is a special technical effect that it is possible to perform skin care by the corresponding LED wavelength after measuring the skin elasticity of the user through the piezoelectric sensor 410 of the fifth embodiment before skin care, then selecting an appropriate LED wavelength according to the user.

In addition, according to a tenth embodiment, the thickness of the adipose tissue layer of the skin of the user may be accurately measured before skin care, and through this, a wavelength tunable light-emitting module 440 of the ninth embodiment is organically combined to control the optimal LED wavelength suitable for the user to perform skin care, and accordingly, there is a technical effect that may maximize the effect of skin care.

In addition, according to an eleventh embodiment, the linear ultrasonic waves may be generated using a linear ultrasound module 460, and after accurately measuring the skin thickness of the user according to the tenth embodiment, the linear ultrasound module 460 is used in consideration of the skin thickness of the user and a depth of treatment or care, and accordingly, there is a special technical effect that may exert an intensive and very optimal care effect.

In addition, according to a twelfth embodiment, the skin thickness of the user may be accurately measured by an ultrasound module 471 and a light detection module 472, and through this, the wavelength tunable light-emitting module 440 of the ninth embodiment is organically combined to control the optimal LED wavelength suitable for the corresponding user and perform skin care, and accordingly, there is a technical effect that may maximize the effect of skin care.

In addition, according to the twelfth embodiment, after accurately measuring the user's skin thickness using the linear ultrasonic module 460 of the eleventh embodiment and using the linear ultrasonic module 460 in consideration of the user's skin thickness and treatment or care depth, and accordingly, there is a special technical effect that may exert an intensive and very optimal care effect.

### DESCRIPTION OF DRAWINGS

FIG. 1 is a perspective view of a facial beauty device according to an embodiment.

FIG. 2 is an exploded perspective view of the facial beauty device according to the embodiment shown in FIG. 1.

FIG. 3 is a detailed view of a circuit board in the facial beauty device according to the embodiment shown in FIG. 2.

FIG. 4A is a first detailed view of a second face body in the facial beauty device according to the embodiment shown in FIG. 2.

FIG. 4B is a second detailed view of the second face body in the facial beauty device according to the embodiment shown in FIG. 2.

FIG. 4C is a third detailed view of the second face body in the facial beauty device according to the embodiment shown in FIG. 2.

FIG. 5 is a partial perspective view of a facial beauty device according to the embodiment.

FIG. 6 is a partial perspective view of a mask sheet and a stimulation providing part in the facial beauty device according to the embodiment shown in FIG. 5.

FIG. 7A is a partial cross-sectional view of a facial mask according to a second embodiment.

FIG. 7B is a partial cross-sectional view of a facial mask according to a third embodiment.

FIG. 7C is a partial cross-sectional view of a facial mask according to a fourth embodiment.

FIG. 8A is a partial cross-sectional view of a facial mask according to a fifth embodiment.

FIG. 8B is a front view of an inner side of a second face body in a facial mask according to a sixth embodiment.

FIG. 9 is a partial cross-sectional view of a facial mask according to a seventh embodiment.

FIG. 10 is a partial cross-sectional view of a facial mask according to an eighth embodiment.

FIG. 11 is a partial cross-sectional view of a facial mask according to a ninth embodiment.

FIG. 12 is a partial cross-sectional view of a facial mask according to a tenth embodiment.

FIGS. 13A and 13B are partial cross-sectional views of a facial mask according to an eleventh embodiment.

FIG. 14 is a partial cross-sectional view of a facial mask according to a twelfth embodiment.

FIG. 15 is a control block diagram of a skin care device according to an embodiment.

FIG. 16 is a control block diagram of a skin care device system including the skin care device according to the embodiment.

### MODES OF THE INVENTION

Hereinafter, the present invention will be described in more detail with reference to the drawings.

The suffixes “module” and “part” for the elements used in the following description are given in consideration of only the ease of creating the specification, and do not impart a particularly important meaning or role by themselves. Therefore, the “module” and “part” may be used interchangeably with each other.

The terms “first,” “second,” etc. May be used herein to describe various elements, but these elements should not be limited by these terms. These terms are only used to distinguish one element from another.

A singular expression includes a plural expression unless the context clearly refers to otherwise.

It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

### EMBODIMENT

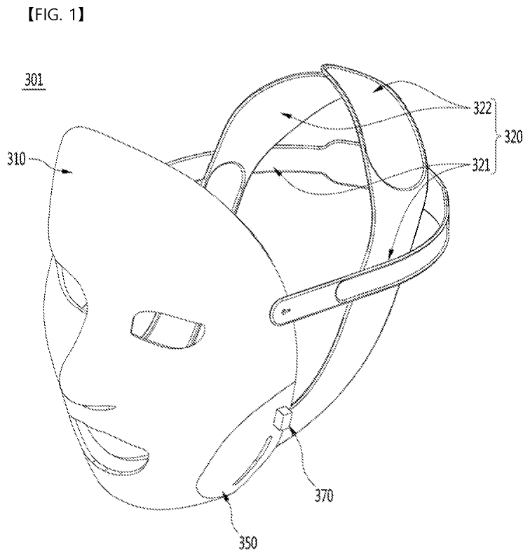

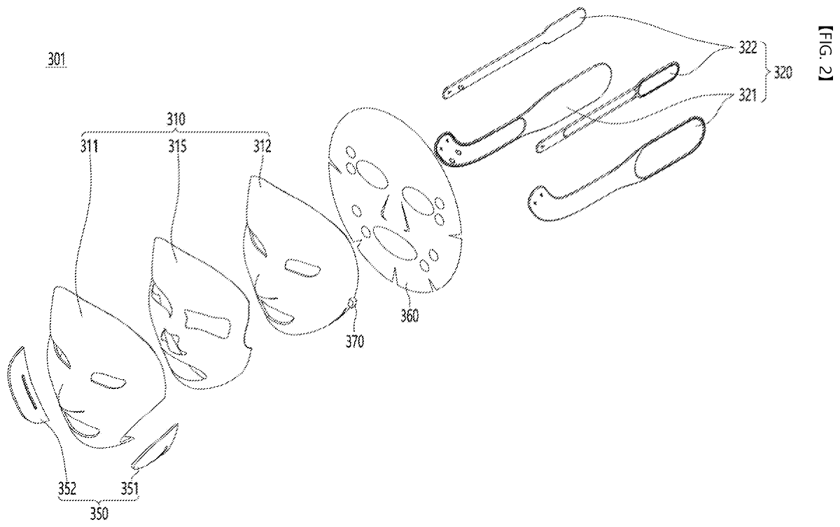

FIG. 1 is a perspective view of a facial beauty device 301 according to an embodiment, and FIG. 2 is an exploded perspective view of the facial beauty device 301 according to the embodiment shown in FIG. 1.

Referring to FIG. 1, the facial beauty device 301 according to the embodiment may include one or more among a face body 310, a mounting member 320, a support part 350, and a power module 370.

For example, the facial beauty device 301 according to the embodiment may include the face body 310, the mounting member 320 coupled to the face body 310, the support part 350 disposed on one side of the face body 310, and a power module 370 disposed on the support part 350. The mounting member 320 may include a first mounting member 321 and a second mounting member 322.

Hereinafter, technical features of the facial beauty device 301 according to the embodiment will be described in detail with reference to FIGS. 2 to 6.

<Face Body>

Referring to FIG. 2, in an embodiment, the face body 310 may include the first face body 311 and the second face body 312, and the circuit board 315 disposed between the first face body 311 and the second face body 312.

For example, in the embodiment, the face body 310 may include the first face body 311 which is flexible and the second face body 312 which is an area in contact with a user's face. The first face body 311 may be referred to as an outer face body, and the second face body 312 may be referred to as an inner face body, but the embodiment is not limited thereto.

The first face body 311 and the second face body 312 are formed of a flexible material, so that there are effects that the facial beauty device 301 according to the embodiment may implement close contact with the face and may be washed with water and waterproof.

For example, the first face body 311 and the second face body 312 may be made of a flexible material such as silicone, thermo plastic elastomer (TPE), or urethane.

In an embodiment, a hardness of the first face body 311 and that of the second face body 312 may be different from each other. For example, the hardness of the second face body 312 disposed close to the user's face may be implemented to be higher than that of the first face body 311.

Accordingly, while maintaining the overall shape in the second face body 312, the lifting effect may be increased by relatively high flexibility in the first face body 311.

According to the embodiment, there is an effect that it is possible to implement close contact with the face, to intensively care for the skin, and to wash with water and be waterproof by applying the flexible material such as silicone, TPE, or urethane to the face body, and at the same time, by the face lifting effect by the mounting member 320 described later.

For example, according to the embodiment, when the user uses the face body, the flexible material is not simply placed on the face, but the face body is lifted by pulling the mounting member that is a stretch bending part to the chin, and at the same time, is in close contact with the face, so that effects such as heat, galvanic, light, etc. may be further maximized as the face body is in direct close contact with the face.

Accordingly, according to the embodiment, it is possible to provide a three-dimensional (3D) flexible mask capable of customized multi-care for each skin area capable of V-line lifting, microcurrent, or galvanic therapy.

<Circuit Board>

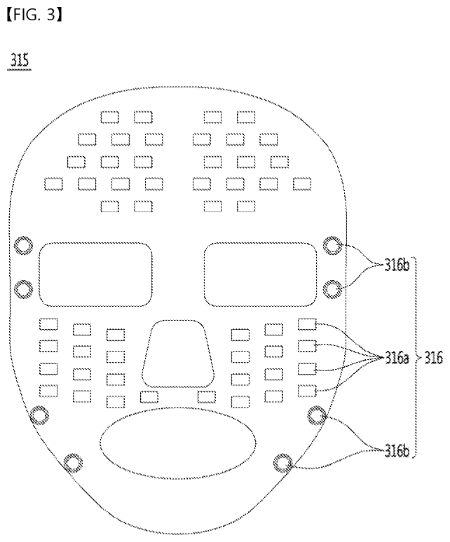

Next, the face body 310 includes the circuit board 315 disposed between the first face body 311 and the second face body 312, and hereinafter, the circuit board 315 will be described with reference to FIG. 3A. In the embodiment, the circuit board 315 may be an FPCB board, but the embodiment is not limited thereto.

As shown in FIG. 3, the embodiment may include a single or a plurality of stimulation providing parts 316 on the circuit board 315.

For example, the stimulation providing part 316 according to the embodiment may include at least one of a light-emitting module 316a, a heating module (not shown), an ion module 316b, or a vibration module (not shown).

The light-emitting module 316a may be disposed at a position corresponding to a wide area of the face, such as cheeks and forehead and may provide skin care effects such as an increase in skin elasticity and a skin regeneration effect.

For example, the light-emitting module 316a may include an LED module or an OLED module. For example, when the light-emitting module 316a includes the LED module, the LED module emits a red wavelength of about 630 nm to increase the dermal density of the facial skin, increase skin brightness, and increase skin elasticity.

In addition, when the light-emitting module 316a emits a blue wavelength, light directly penetrates to a depth of about 8 to 10 mm inside the skin due to a close contact structure, so that skin regeneration, acne, etc. may be improved depending on the wavelength.

In addition, since the stimulation providing part 316 of the embodiment has a predetermined resistance (not shown), it is possible to implement a thermal care effect by a heating function.

In addition, the light-emitting module 316a of the embodiment may include a heating element that converts power supplied from the power module 370 into heat. The facial beauty device 301 may include a plurality of light-emitting modules 316a, and when the light-emitting module 316a generates heat, there is an effect capable of promoting the absorption of ingredients contained in a contacted mask sheet 360 into the skin.

In addition, the stimulation providing part 316 according to the embodiment may include the light-emitting module 316a and a separate heating module (not shown). For example, the heating module may include a heating element, and a heating temperature may have a temperature between 24 and 39° C., at which it is easy to absorb into the skin and moisturizing ingredients may be absorbed into the skin. Through the heat generation, it is possible to promote absorption of nutritional ingredients, moisturizing ingredients, and the like contained in the mask sheet 360 described later into the user's face.

The heating element according to the embodiment may be a thermoelectric element having cooling and heating functions. The thermoelectric element applied in the embodiment may be cooled or heated using a Peltier effect that is a phenomenon in which heat is absorbed or generated by a current. The Peltier effect is a phenomenon in which when two types of metal ends are connected and current is passed through them, one terminal absorbs heat and the other terminal generates heat according to a direction of the current. The thermoelectric element of the embodiment may obtain a Peltier device that absorbs heat and generates heat with high efficiency by using a semiconductor such as bismuth tellurium having a different electrical conduction method instead of two kinds of metals. Such a Peltier device may switch between heat absorption and heat generation according to the direction of the current, and heat absorption amount and heat generation amount may be adjusted according to an amount of the current.

In addition, the stimulation providing part 316 of the embodiment may provide a galvanic effect of increasing penetration into the facial skin by ionizing active ingredients through a microcurrent by having an ion module 316b, thereby improving eye wrinkles or chin wrinkles.

For example, the ion module 316b may supply positive ions or negative ions through an alternating current or may supply positive ions or negative ions through a galvanic current.

In particular, when the ion module 316b supplies positive ions or negative ions to the skin through the galvanic current, there is an advantage of supplying positive ions or negative ions to the skin while minimizing skin irritation.

For example, the stimulation providing part 316 of the embodiment may include a plurality of ion modules 316b, and when the stimulation providing part 316 includes the plurality of ion modules 316b, a current intensity may be individually set for each of the plurality of ion modules 316b. Through this, by adjusting the current intensity according to the characteristics of the skin on which the ion module 316b is disposed, there is an advantage that customized care for each skin area is possible.

For example, the stimulation providing part 316 includes eight ion modules 316b, and four ion modules 316b may be disposed in a first region, for example, around eye rims, and the remaining four ion modules 316b may be disposed in a second region, for example around the chin.

In addition, the stimulation providing part 316 of the embodiment may be provided with a vibration module (not shown) to increase the promotion of penetration of the active ingredients into the skin, thereby improving eye wrinkles or chin wrinkles.

The vibration module may be in direct or indirect contact with the user's skin to transmit vibration. The vibration module may induce vibration by a vibrator that vibrates by an electric signal.

For example, the stimulation providing part 316 of the embodiment may include a plurality of vibration modules, and when the stimulation providing part 316 includes the plurality of vibration modules, the current intensity and the number of vibrations may be individually set for each of the plurality of vibration modules. Through this, by adjusting at least one of vibration intensity and the number of vibrations according to the characteristics of the skin on which the vibration module is disposed, there is an advantage that customized care for each skin area is possible.

<Power Module>

Referring back to FIG. 2, the embodiment may include the power module 370 electrically connected to the stimulation providing part 316, and the power module 370 may be connected to a predetermined power supply line (not shown).

In the embodiment, a communication module (not shown) may be further disposed on the face body 310, but the embodiment is not limited thereto.

The power module 370 may be disposed on the second face body 312, but the embodiment is not limited thereto, and may be disposed on the circuit board 315 or may be disposed on the support part 350 described later.

The power module 370 may include an input port (not shown) for receiving power from the outside, and power supplied through the input port may be supplied to the stimulation providing part 316 such as a light-emitting module 316a or an ion module 316b. For example, the input port may be a port to which a universal serial bus (USB) terminal is connected.

Alternatively, the power module 370 may include a battery connecting part (not shown) to which a battery is detached/attached and may supply power supplied from the battery connected to the battery connecting part to the ion module 316b or the light-emitting module 316a.

As described above, the power module 370 may supplied with power from the outside in various ways. The above example is merely illustrative for convenience of description, and thus it is reasonable that the embodiment is not limited thereto.

In an embodiment, the power module 370 may further include a power button (not shown) for controlling power on/off of the facial beauty device 301. The user may turn on or off the power of the facial beauty device 301 rough the power button.

When the power of the facial beauty device 301 is controlled to be turned on through the power button, the power module 370 may supply power to at least one of the ion module 316b and the light-emitting module 316a, and when the power of the facial beauty device 301 is controlled to be turned off through the power button, the power supplied to the ion module 316b and the light-emitting module 316a may be cut off.

The mask sheet 360 described later includes formulation ingredients in a predetermined liquid state, and the formulation ingredients may flow downward by gravity.

Accordingly, the power module 370 of the embodiment is not positioned on a lower surface of the face body 310 so as not to come into contact with the formulation ingredients of the mask sheet 360 but is disposed on a side surface to prevent an electrical short-circuit.

<Mounting Member>

Referring to FIG. 2, in the embodiment, the mounting member 320 may be coupled to the face body 310. The mounting member 320 may be referred to as a mounting band, but the embodiment is not limited thereto.

In the embodiment, the mounting member 320 may be provided in single or plural. For example, the mounting member 320 may include the first mounting member 321 coupled to the first region of the second face body 312, for example, a center edge and the second mounting member 322 coupled to the second region of the second face body 312, for example, a lower edge.

According to the embodiment, a sensation effect of V-line lifting may be maximized by pressing the V-line on the face when wearing the facial mask.

For example, according to the embodiment, it is possible to implement close contact with the face by applying the flexible material such as silicone, TPE, or urethane to the face body 310, and at the same time, by lifting the face by including two bands with a band next to the eye as the first mounting member 321 and a stretching band of the chin as the second mounting member 322, thereby improving the V-line lifting effect.

In addition, according to the embodiment, when in use of the face body, the flexible material is not simply placed on the face, but the face body of areas of the chin and eye is lifted by the mounting member 320, and at the same time, is in close contact with the face, so that effects such as heat, galvanic, light, etc. may be further maximized as the face body is in direct close contact with the face.

<Frame>

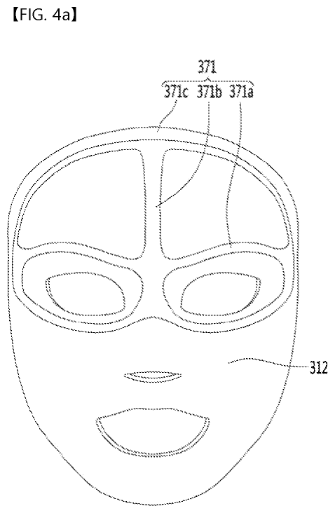

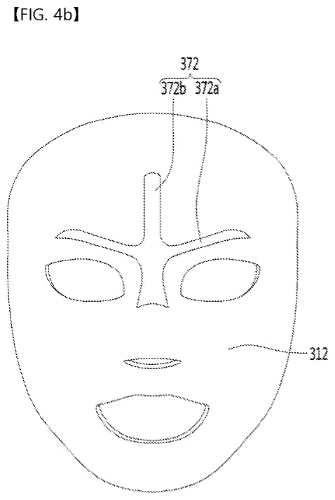

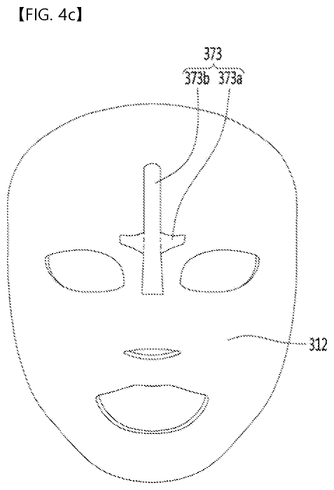

Next, referring to FIGS. 4A to 4C, the embodiment is provided with a frame on the face body, so that there is a technical effect that it is possible to prevent excessive pressing of the forehead, nose, and eye area having some curved surfaces of the face when the face body is in close contact with the face, and to protect the curved surface of the face while being in close contact with the curved surface of the face.

For example, FIG. 4A is a first detailed view of the second face body 312 in the facial beauty device according to the embodiment shown in FIG. 2.

The embodiment may include a first frame 371 on the second face body 312. The first frame 371 may be formed of a resin material having high elasticity and chemical resistance, but the embodiment is not limited thereto. The first frame 371 is illustrated to be disposed on the second face body 312, but the embodiment is not limited thereto and may be disposed on the circuit board 315.

The first frame 371 may include a first-first frame 371a disposed around the eye area and a first-third frame 371c disposed around the forehead.

In addition, the first frame 371 may include a first-second frame 371b disposed between the first-first frame 371a and the first-third frame 371c.

Accordingly, according to an embodiment, there is a technical effect that it is possible to minimize the compression of a bone by the first-third frames 371c disposed around the forehead, and to protect important areas of the face such as eyes and forehead by the first-first frame 371a disposed around the eye area when a chin area of the flexible face body is in close contact with the face.

In addition, it is possible to enhance a protection function of the forehead area and the eye area while strengthening a frame structure by the first-second frame 371b disposed between the first-first frame 371a and the first-third frame 371c.

According to the embodiment, the embodiment is provided with the first frame 371 on the face body, so that there is a technical effect that it is possible to more firmly prevent excessive pressing of the forehead, nose, and eye area having some curved surfaces of the face when the face body is in close contact with the face, and to protect the curved surface of the face while being in close contact with the curved surface of the face.

Next, FIG. 4B is a second detailed view of the second face body 312 in the facial beauty device according to the embodiment shown in FIG. 2.

The second frame 372 may include a second-first frame 372a disposed around the eyes and a second-second frame 372b disposed around the forehead.

According to the embodiment, it is possible to protect important areas of the face such as eyes and forehead by the second-first frame 372a disposed around the eye area when the chin area of the flexible face body is in close contact with the face, and it is possible to enhance the protection function of the forehead area while strengthening the frame structure by the second-second frames 372b disposed around the forehead.

According to the embodiment, the embodiment is provided with the second frame 372 on the face body, so that there is a technical effect that it is possible to more firmly prevent excessive pressing of the forehead, nose, and eye area having some curved surfaces of the face when the face body is in close contact with the face, and to protect the curved surface of the face while being in close contact with the curved surface of the face.

Next, FIG. 4C is a third detailed view of the second face body 312 in the facial beauty device according to the embodiment shown in FIG. 2.

The third frame 373 may include a third-first frame 373a disposed around the eyes and a third-second frame 373b disposed around the forehead.

A horizontal width of the third-first frame 373a may be smaller than a vertical width of the third-second frame 373b, but the embodiment is not limited thereto.

According to the embodiment, it is possible to protect important areas of the face such as eyes and forehead by the third-first frame 373a disposed around the eye area when the chin area of the flexible face body is in close contact with the face, and it is possible to enhance the protection function of the forehead area while strengthening the frame structure by the third-second frames 373b disposed around the forehead.

According to the embodiment, the embodiment is provided with the third frame 373 on the face body, so that there is a technical effect that it is possible to more firmly prevent excessive pressing of the forehead, nose, and eye area having some curved surfaces of the face when the face body is in close contact with the face, and to protect the curved surface of the face while being in close contact with the curved surface of the face.

<Support Part>

Referring back to FIG. 2, the embodiment may include the support part 350 disposed on the face body 310.

For example, the support part 350 may be disposed on the first face body 311, and the support part 350 may be formed of a resin having high elasticity and chemical resistance, or the like through injection, but the embodiment is not limited thereto.

The support part 350 may include a first support part 351 disposed on one lower side of the first face body 311 and a second support part 352 disposed on the other lower side of the first face body 311.

The support part 350 may prevent the mounting member or the face body from being broken or damaged when the mounting member 320 is stretched for a lifting effect.

The ion module 316b described above may be disposed on the support part 350 to implement the galvanic effect by microcurrent injection.

<Mask Sheet>

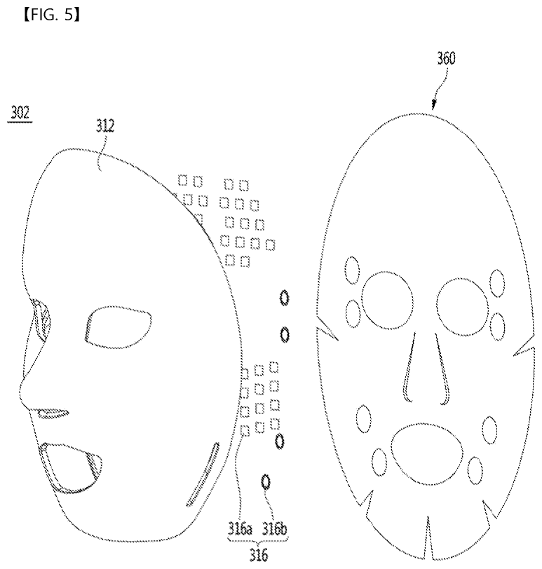

Next, FIG. 5 is a partial perspective view of a facial beauty device 302 according to the embodiment shown in FIG. 2 and is a perspective view of the second face body 312, the mask sheet 360, and the stimulation providing part 316.

In FIG. 5, the stimulation providing part 316 is conceptually disposed for the purpose of describing the contents and it is not limited to being disposed at the position shown in FIG. 5.

The facial mask 302 according to the embodiment may further include the mask sheet 360 that may be used together with a corresponding device inside the second face body 312 disposed on the user's face side.

Accordingly, according to the embodiment, there is a technical effect capable of increasing the skin efficacy effect through the multi-use of the formulation and the beauty device by wearing together with the mask sheet 360 containing the formulation.

The mask sheet 360 according to the embodiment may be used together with a sheet material having excellent light and heat transmittance and excellent adhesion, unlike an opaque material such as a commonly used PET material, or the like. For example, the mask sheet 360 may be used together with a sheet material having excellent light and heat transmittance, such as a cellulose-based lyocell, rayon-based hydrogel, bio-cellulose, a material with improved transmittance by modifying a rayon-based material, or the like, but the embodiment is not limited thereto.

In addition, in case of the formulation included in the mask sheet 360 in the embodiment, since the transparency may be hindered depending on a particle size of an emulsion, it may be not a formulation that is an opaque emulsion type or has a large particle size as much as possible but a formulation that has a high absorption rate due to a feeling of warmth while having transparency.

The mask sheet 360 of the embodiment may be a material containing formulation ingredients such as nutritional ingredients and moisturizing ingredients for skin care, and the material may be made of a nonwoven fabric such as cotton or cloth, a hydrogel, or the like.

According to the embodiment, the mask sheet 360 may include a microneedle containing an effective ingredient. A material of the microneedle may be a solid-type such as a metal material, silicone, and the like, or may be a soluble-type that is dissolved when applied to the skin using a self-degradable material, a biodegradable material, or the like. A wrinkle improvement and whitening efficacy material such as botox, retinol, hyaluronic acid, and the like may be included in the microneedle. When the microneedle is in close contact with the skin, the efficacy material may penetrate deep into the skin. When the facial beauty devices 301 and 302 proposed in the embodiment are worn on the face, the microneedles may be smoothly attached and absorbed by making them to be in close contact with and to press the face.

Next, FIG. 6 is a partial perspective view of the mask sheet 360 and the stimulation providing part 316 in the facial beauty device according to the embodiment shown in FIG. 5. In FIG. 6, the stimulation providing part 316 is conceptually disposed for description and is not limited to being disposed at the position shown in FIG. 6.

In the embodiment, there is an effect that the mask sheet 360 has a predetermined opening 360h so that a microcurrent electrode of the stimulation providing part 316 may be energized to the face even after the mask sheet 360 is attached.

For example, in the embodiment, the mask sheet 360 may include a plurality of openings 360h. For example, the mask sheet 360 may include a first opening 360h1 corresponding to an eye and a second opening 360h2 corresponding to a mouth.

In particular, the mask sheet 360 according to the embodiment may include the stimulation providing part 316, for example, a third opening 360h3 corresponding to the ion module 316b.

As described above, the mask sheet having a predetermined opening corresponding to the position of the stimulation providing part 316 may be referred to as a dedicated product used only for the production of the facial masks 301 and 302.

In addition, as described above, the mask sheet having a predetermined opening corresponding to the position of the stimulation providing part 316 may be referred to as a dedicated product used only for the use of the facial masks 301 and 302.

In this case, a second diameter D2 of the third opening 360h3 of the mask sheet 360 may be larger than a first diameter D1 of the ion module 316b.

Accordingly, the embodiment may implement a mask sheet form in which a microcurrent of the ion module 316b may be directly energized to the skin.

Accordingly, in the embodiment, there is a special technical effect that the mask sheet 360 has the predetermined opening so that the microcurrent electrode of the stimulation providing part may be energized to the face even after the mask sheet 360 is attached.

According to the embodiment, there are technical effects as follows.

First, according to the embodiment, it is possible to provide the three dimensional (3D) flexible mask capable of customized multi-care for each skin area.

In addition, according to the embodiment, there is an effect that it is possible to implement close contact with the face, to intensively care for the skin, and to wash with water and be waterproof by applying the flexible material such as silicone, TPE, or urethane to the face body, and at the same time, by the face lifting effect by the mounting member 320 described later.

In addition, the embodiment is provided with the frame on the face body, so that there is a technical effect that it is possible to prevent excessive pressing of the forehead, nose, and eye area having some curved surfaces of the face when the face body is in close contact with the face, and to protect the curved surface of the face while being in close contact with the curved surface of the face.

In addition, according to the embodiment, there is a technical effect capable of increasing the skin efficacy and effect through the multi-use of the formulation and the beauty device by wearing together with the mask sheet 360 containing the formulation.

In addition, in the embodiment, the mask sheet 360 has a predetermined opening so that the microcurrent electrode of the stimulation providing part may be energized to the face even after the mask sheet 360 is attached.

Hereinafter, technical features of second to twelfth embodiments will be described.

### Second Embodiment

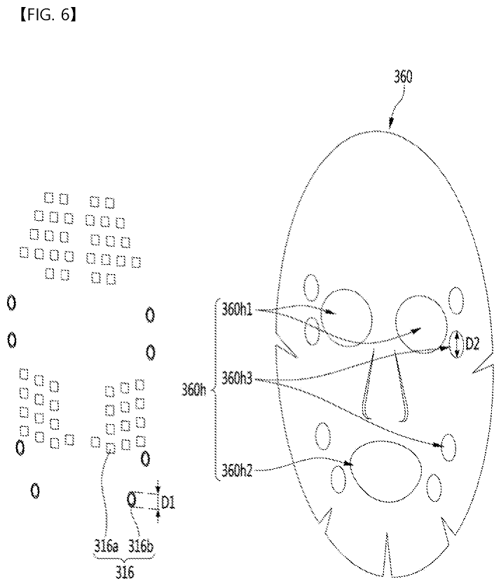

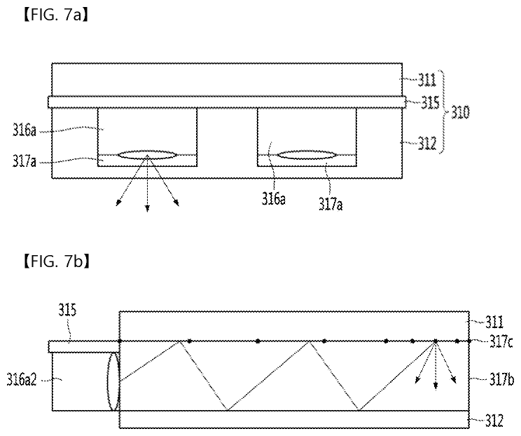

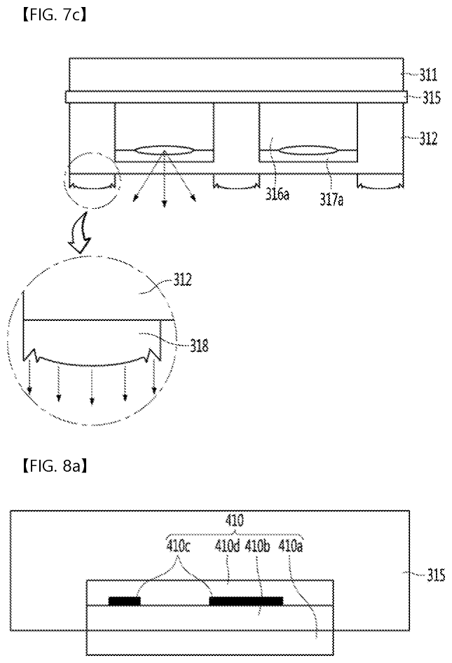

First, FIG. 7A is a partial cross-sectional view of a facial mask according to a second embodiment.

The second embodiment may apply the technical features of the facial masks 301 and 302 according to the above-described embodiment, and hereinafter, the main features of the second embodiment will be mainly described.

Referring to FIG. 7A, the facial mask in the second embodiment may include a face body 310, a first light-emitting module 316a disposed on the face body 310, and a first diffusion plate 317a disposed on the first light-emitting module 316a.

The face body 310 may include a first face body 311, a second face body 312, and a circuit board 315 between the first face body 311 and the second face body 312.

In the second embodiment, the first light-emitting module 316a may be a top view light-emitting device that emits light upward, but the embodiment is not limited thereto.

In the related art, it is difficult to uniformly irradiate LED light over the entire face, and accordingly, there is a problem that a uniform care effect cannot be obtained over the entire face.

In addition, when LED elements are densely disposed so as to correspond to the entire face, not only it is difficult to implement a flexible mask due to contact or interference between the LED elements, but also there is a problem of a technical contradiction that overheating occurs.

Accordingly, the second embodiment includes the first diffusion plate 317a disposed on the first light-emitting module 316a to diffuse light emitted from the first light-emitting module 316a, so that there is a technical effect that it is possible to increase the overall uniform face care effect by evenly irradiating LED light to the entire facial skin even though the light-emitting module is disposed in an appropriate range.

### Third Embodiment

Next, FIG. 7B is a partial cross-sectional view of a facial mask according to a third embodiment.

The third embodiment may apply the technical features of the facial masks 301 and 302 according to the above-described embodiment, and hereinafter, the main features of the third embodiment will be mainly described.

Referring to FIG. 7B, the facial mask in the third embodiment may include a second light-emitting module 316a2 disposed on the circuit board 315, a second diffusion plate 317b disposed on a side surface of the second light-emitting module 316a2, and a reflective material coating layer 317c disposed above the second diffusion plate 317b.

The reflective material coating layer 317c may be disposed to be spaced apart in plural. For example, as the reflective material coating layer 317c is far from the second light-emitting module 316a2, the separation distance may be closer, but the embodiment is not limited thereto.

The second diffusion plate 317b may include a flexible light guide sheet. In the third embodiment, the second light-emitting module 316a2 may be a side view LED that emits light in a side direction, but the embodiment is not limited thereto.

According to the third embodiment, light emitted from the second light-emitting module 316a2 is diffused by the second diffusion plate 317b disposed on the side surface of the second light-emitting module 316a2 and the reflective material coating layer 317c, so that there is a technical effect that it is possible to increase the overall uniform face care effect by evenly irradiating LED light to the entire facial skin even though the light-emitting module is disposed in an appropriate range.

### Fourth Embodiment

Next, FIG. 7C is a partial cross-sectional view of a facial mask according to a fourth embodiment.

The fourth embodiment may apply the technical features of the facial masks 301 and 302 according to the above-described embodiment and the second embodiment, and hereinafter, the main features of the fourth embodiment will be mainly described.

Referring to FIG. 7C, the facial mask in the fourth embodiment may include the first light-emitting module 316a disposed on the circuit board 315, the first diffusion plate 317a disposed on the first light-emitting module 316a, and a diffraction optical pattern 318 disposed between the first diffusion plates 317a.

The diffraction optical pattern 318 is illustrated to protrude upward from the second face body 312, but the embodiment is not limited thereto, and may also be disposed in a height the same as that of the second face body 312.

The fourth embodiment includes the diffraction optical pattern 318 disposed between the first diffusion plates 317a, so that it is possible to convert a direction of light emitted from the first light-emitting module 316a to diffuse in a region close to the facial skin into parallel light perpendicular to the facial skin.

In the embodiment, a position of the diffraction optical pattern 318 is not limited to between the first diffusion plates 317a, and the diffraction optical pattern 318 may be widely provided under the first diffusion plate 317a as necessary.

Accordingly, according to the fourth embodiment, an LED light source emitted from the first light-emitting module 316a may be uniformly concentrated and irradiated on the face by the diffraction optical pattern 318 disposed between or under the first diffusion plate 317a, and thus it is possible to improve an effect of increasing the dermal density of the facial skin, increasing skin brightness, and increasing skin elasticity due to excellent penetration into the facial skin.

### Fifth Embodiment

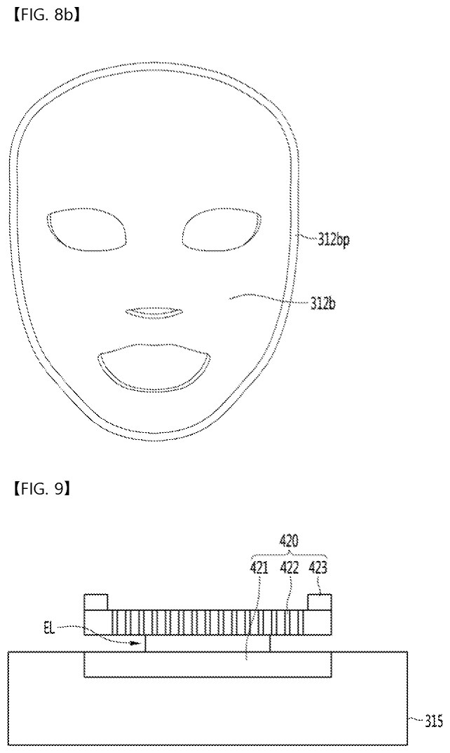

Next, FIG. 8A is a partial cross-sectional view of a facial mask according to a fifth embodiment.

The fifth embodiment may apply the facial masks 301 and 302 according to the above-described embodiment and the above-described embodiments, and hereinafter, the main features of the fifth embodiment will be mainly described.

Referring to FIG. 8A, the facial mask in the fifth embodiment may include a piezoelectric sensor 410 disposed on a circuit board 315.

In the fifth embodiment, the piezoelectric sensor 410 may include a flexible substrate 410a, a piezoelectric thin film layer 410b on the flexible substrate 410a, an electrode 410c on the piezoelectric thin film layer 410b, and a protective layer 410d on the electrode 410c.

The piezoelectric thin film layer 410b may include an inorganic material having a perovskite structure. For example, the piezoelectric thin film layer 410b may include a material such as BaTiO3, LiNbO3, LiTaO3, KNbO3, and the like, but the embodiment is not limited thereto.

A facial beauty device according to the related art has a problem that treatment or care is uniformly performed with a function set in the product itself rather than considering skin elasticity of the user to be treated.

In particular, in the related art, it is difficult to check in real time how much skin elasticity is improved during skin care.

Accordingly, the fifth embodiment includes the piezoelectric sensor 410 having the piezoelectric thin film layer 410b disposed on the flexible substrate 410a, and accordingly, there is a special technical effect that it is possible to measure skin elasticity in real time during skin care.

For example, according to the fifth embodiment, there is a special technical effect that it is possible to measure skin elasticity in real time during skin care by detecting minute changes in skin elasticity through the piezoelectric thin film layer 410b and the electrode 410c.

In order to increase the sensitivity of the skin elasticity change, a signal amplifying unit, a signal filtering unit, and the like may be further included.

### Sixth Embodiment

FIG. 8B is a front view of an inner side 312b of a second face body in a facial mask according to a sixth embodiment.

The sixth embodiment may apply the facial masks 301 and 302 according to the above-described embodiment and the embodiments, and hereinafter, the main features of the sixth embodiment will be mainly described.

According to the related art, a mask for facial care is used spaced apart from the face, and even though the mask is to be brought into close contact with the face, it is difficult to use the mask in close contact with the face.

Meanwhile, referring to FIG. 8B, the sixth embodiment may include the adhesive layer 312bp at the edge of the inner side 312b of the second face body to increase the adhesion between the facial mask and the facial skin, thereby increasing the effect of skin care.

For example, in the sixth embodiment, the adhesion to the skin is increased by disposing the adhesive layer 312bp made of an acrylic monomer material with high adhesion and biocompatibility at the edge of the second face body, and accordingly, the adhesion between the face mask and the facial skin is increased, thereby increasing the effecting of skin care.

In particular, according to the sixth embodiment, the mask sheet 360 is positioned at the inner side 312b of the second face body, but the mask sheet 360 including a formulation is more closely adhered to the face by disposing the adhesive layer 312bp at the edge of the second face body, and accordingly, the effect of skin care is increased by active ingredients, and the adhesion between the face mask and the facial skin is increased, thereby increasing the effect of skin care.

### Seventh Embodiment

Next, FIG. 9 is a partial cross-sectional view of a facial mask according to a seventh embodiment.

The seventh embodiment may apply the facial masks 301 and 302 according to the above-described embodiment and the embodiments, and hereinafter, the main features of the seventh embodiment will be mainly described.

Referring to FIG. 9, the facial mask according to the seventh embodiment may include a contact time sensing device 420 disposed on the circuit board 315.

In the seventh embodiment, the contact time sensing device 420 may include a monitor module 421, a plurality of electrical contacts 422, and a plurality of ports 423. The plurality of electrical contacts 422 may be connected to the monitor module 421 by a power line EL.

According to the embodiment, face care may be performed using the mask sheet 360 containing the formulation.

At this time, the formulation contains moisture, and the moisture decreases as it is used.

Accordingly, in the seventh embodiment, here is a special technical effect that can check in real time the degree of use of the mask sheet 360 and the progress of the skin care time by measuring a moisture content of the formulation contained in the mask sheet 360.

For example, in the seventh embodiment, the contact time sensing device 420 may maintain a voltage on a first electrical contact point of the port 423 and detect a short-circuit between the first electrical contact point and a second electrical contact point of the port, and when the monitor module 421 detects the short-circuit, the contact time sensing device 420 may determine that the formulation of the mask sheet 360 remains sufficiently, and when the short-circuit is not detected, the contact time sensing device 420 may determine in real time that the formulation is insufficient.

Through this, in the seventh embodiment, there is a special technical effect that it is possible to check the degree of use of the mask sheet 360, the progress of skin care time, and the like in real time by accurately measuring the moisture content of the cosmetic formulation contained in the mask sheet 360 through the contact time sensing device 420.

### Eighth Embodiment

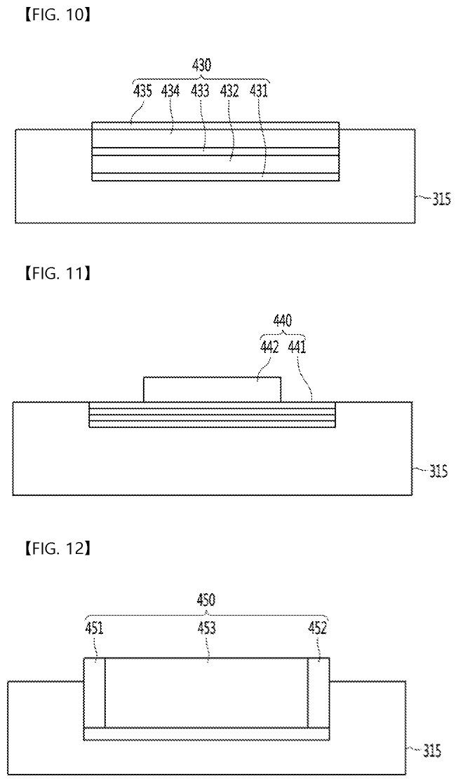

Next, FIG. 10 is a partial cross-sectional view of a facial mask according to an eighth embodiment.

The eighth embodiment may apply the facial masks 301 and 302 according to the above-described embodiment and the embodiments, and hereinafter, the main features of the eighth embodiment will be mainly described.

According to the related art, a light-emitting device and a vibration device for skin care are applied in a form of individual devices.

However, when the light-emitting device and the vibration device are disposed as separate devices, there is a technical contradiction that uniform care may not be implemented over the entire face because the light-emitting device may not be disposed in an area where the vibration device is disposed.

In order to overcome this technical contradiction, as shown in FIG. 10, the facial mask in the eighth embodiment may include an optical vibration composite element 430 disposed on the circuit board 315.

For example, the optical vibration composite element 430 in the eighth embodiment may include a first electrode layer 431 disposed on the circuit board 315, a flexible light-emitting layer 432 disposed on the first electrode layer 431, a second electrode layer 433 disposed on the flexible light-emitting layer 432, a flexible ultrasonic layer 434 disposed on the second electrode layer 433, and a third electrode layer 435 disposed on the flexible ultrasonic layer 434.

The first electrode layer 431 and the third electrode layer 435 may be an anode, and the second electrode layer 433 may be a common cathode.

DC power may be applied to the flexible light-emitting layer 432, and AC power may be applied to the flexible ultrasonic layer 434.

The flexible light-emitting layer 432 may be an OLED, and the flexible ultrasonic layer 434 may be a flexible piezo coating layer, but the embodiment is not limited thereto.

According to the eighth embodiment, there is a technical effect that it is possible to simultaneously implement light and ultrasonic waves for facial care by providing the optical vibration composite element 430 including the flexible light-emitting layer 432 and the flexible ultrasonic layer 434.

In particular, according to the eighth embodiment, as light and ultrasonic waves may be simultaneously implemented by one composite device 430, the optical vibration composite element 430 may be densely disposed over the entire face, and accordingly, there is a special technical effect that it is possible to implement very uniform care over the entire face.

### Ninth Embodiment

Next, FIG. 11 is a partial cross-sectional view of a facial mask according to a ninth embodiment.

The ninth embodiment may apply the facial masks 301 and 302 according to the above-described embodiment and the embodiments, and hereinafter, the main features of the ninth embodiment will be mainly described.

According to the related art, when an LED is used in a skin care apparatus, a single wavelength LED is used or an LED capable of emitting individual wavelengths is used even though an LED of a plurality of various wavelengths are used.

Accordingly, the number of LEDs that may be mounted is limited in the conventional LED mask, and there was a limitation that an emission wavelength may not be used in various ways according to needs of a user.

Referring to FIG. 11, the facial mask according to the ninth embodiment may include a wavelength tunable light-emitting module 440. The wavelength tunable light-emitting module 440 may include a piezoelectric substrate 441 disposed on the circuit board 315 and a wavelength tunable light-emitting device 442 disposed on the piezoelectric substrate 441.

For example, as in the fifth embodiment described above, the skin elasticity of the user may be measured by including the piezoelectric sensor 410 including the piezoelectric thin film layer 410b disposed on the flexible substrate 410a.

According to the ninth embodiment, there is a special technical effect that it is possible to perform skin care by the corresponding LED wavelength after measuring the skin elasticity of the user through the piezoelectric sensor 410 of the fifth embodiment before skin care, then selecting an appropriate LED wavelength according to the user.

Alternatively, it may be varied to an appropriate wavelength according to a care purpose of the user. For example, in the case of atopic dermatitis care, a wavelength of about 617 nm may be effective, and in the case of acne care, a wavelength of 630 nm may be effective.

In the ninth embodiment, a wavelength of the wavelength tunable light-emitting device 442 may be varied according to a change in the phenomenon of the piezoelectric substrate 441.

For example, according to the ninth embodiment, a voltage is applied to the piezoelectric substrate 441 to generate mechanical stress, and the mechanical stress may change a wavelength of output light of the wavelength tunable light-emitting device 442, and the piezoelectric substrate 441 may use piezoelectric ceramic, crystal, or the like, but the embodiment is not limited thereto.

For example, the piezoelectric substrate 441 includes a first piezoelectric layer (not shown) and a second piezoelectric layer (not shown) disposed on the first piezoelectric layer, and when the second piezoelectric layer expands in the horizontal direction in a state in which the first piezoelectric layer is not deformed, the piezoelectric substrate 441 may be bent in a convex shape toward thereabove.

Bending occurs also in the wavelength tunable light-emitting device 442 disposed thereon by bending the piezoelectric substrate 441 as described above.

In this case, an active layer (not shown) is formed on the wavelength tunable light-emitting device 442, and expansion and deformation occur in the active layer of the light-emitting device 442 as well. The light-emitting device 442 generates spontaneous polarization according to a stacked structure of atoms composing a compound semiconductor and piezoelectric polarization due to lattice mismatch between a well layer and a barrier layer when the active layer is grown.

When expansion and deformation occur in the active layer due to the convex bending, the polarization effect of the active layer decreases and carriers (electrons and holes) capable of emission recombination increase, and accordingly, there is a characteristic that the emission wavelength shifts to a shorter wavelength as the emission energy increases.

On the other hand, bending occurs so that the piezoelectric substrate 441 has a convex shape toward therebelow, the active layer of the light-emitting device 442 is contracted and deformed in a plane direction, and the polarization phenomenon inside the active layer is intensified and carriers (electrons and holes) capable of emission recombination are reduced, and accordingly, the emission wavelength may shift in a long wavelength direction as the emission energy decreases.

As described above, according to the ninth embodiment, there is a special technical effect that it is possible to perform skin care by the corresponding LED wavelength after measuring the skin elasticity of the user through the piezoelectric sensor 410 of the fifth embodiment before skin care, then selecting an appropriate LED wavelength according to the user.

### Tenth Embodiment

Next, FIG. 12 is a partial cross-sectional view of a facial mask according to a tenth embodiment.

The tenth embodiment may apply the facial masks 301 and 302 according to the above-described embodiment and the embodiments, and hereinafter, the main features of the tenth embodiment will be mainly described.

Referring to FIG. 12, the facial mask according to the tenth embodiment may include a skin thickness measurement element 450.

In the tenth embodiment, the skin thickness measurement element 450 may include one or more ultrasonic transducers 453 disposed on the circuit board 315 and one or more RF electrodes 451 and 452 disposed on both sides of the ultrasonic transducer 453.

According to the tenth embodiment, a thickness of an adipose tissue layer of the skin of the user may be accurately measured before skin care, and through this, the wavelength tunable light-emitting module 440 of the ninth embodiment is organically combined to control an optimal LED wavelength suitable for the user to perform skin care, and accordingly, there is a technical effect that may maximize the effect of skin care.

For example, in the tenth embodiment, a through hole (not shown) may be provided in a part of the second face body 312 and a part of the mask sheet 360 so that the skin thickness measurement element 450 may be in contact with the skin of the user, but the embodiment is not limited thereto.

For example, in the tenth embodiment, the ultrasonic transducer 453 of the skin thickness measurement element 450 and one or more RF electrodes may be coupled to a surface of a skin layer of the user with a predetermined level of force.

For example, electrical impedance of an adipose tissue segment of the skin of the user disposed between a first electrode 451 and a second electrode 452 may be measured by applying a preset voltage between the first electrode 451 and the second electrode 452 placed on the surface of the skin of the user.

For example, a current applied to the first electrode 451 forms a current path along each thickness of the skin layer of the user to flow in the skin tissue, and it is possible to determine the conductivity or impedance of the adipose tissue segment of the user by measuring the total current at an electrode-skin surface coupling point, and through this, a skin thickness of an adipose tissue of the user may be measured.

Accordingly, according to the tenth embodiment, the thickness of the adipose tissue layer of the skin of the user may be accurately measured before skin care, and through this, the wavelength tunable light-emitting module 440 of the ninth embodiment is organically combined to control the optimal LED wavelength suitable for the user to perform skin care, and accordingly, there is a technical effect that may maximize the effect of skin care.

### Eleventh Embodiment

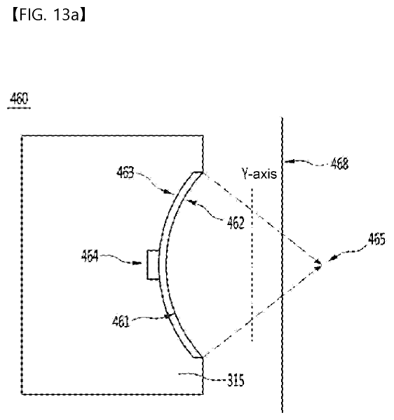

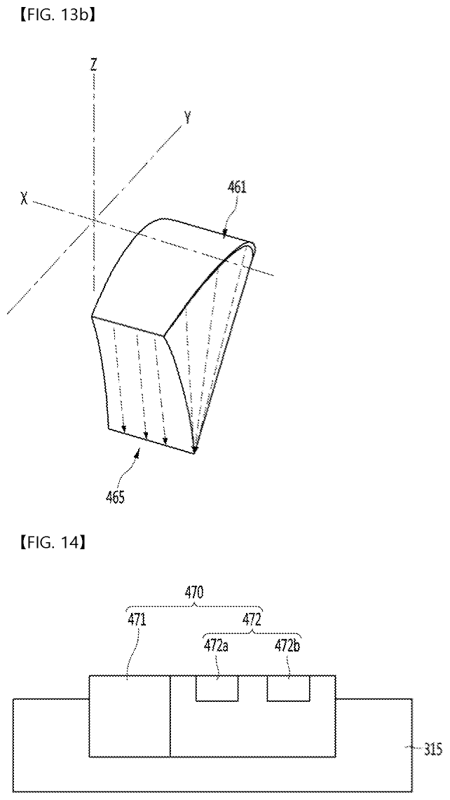

Next, FIGS. 13A and 13B are partial cross-sectional views of a facial mask according to an eleventh embodiment.

The eleventh embodiment may apply the facial masks 301 and 302 according to the above-described embodiment and the embodiments, and hereinafter, the main features of the eleventh embodiment will be mainly described.

Referring to FIG. 13A, the facial mask in the eleventh embodiment may include a linear ultrasound module 460.

For example, the linear ultrasound module 460 of the eleventh embodiment includes a cylindrical conversion element 461 disposed on the circuit board 315 and an imaging element 464 disposed on the cylindrical conversion element 461.

For example, as described above, according to the tenth embodiment, the thickness of the adipose tissue layer of the skin of the user may be accurately measured before skin care.

Thereafter, since it is possible to generate linear ultrasonic waves using the linear ultrasound module 460 of the eleventh embodiment, there is a special technical effect that may exert an intensive and very optimal care effect in consideration of the skin thickness of the user.

For example, referring to FIG. 13A, the linear ultrasound module 460 of the eleventh embodiment may include the cylindrical conversion element 461 and the imaging element 464, and the cylindrical conversion element 461 may have a concave surface 462 and a convex surface 463.

The cylindrical conversion element 461 may be cut opened to show the release of energy from the concave surface 462 to a treatment area 465 in a user skin surface 468.

Referring to FIG. 13B, the ultrasound generated by the linear ultrasound module 460 in the eleventh embodiment may include a spherical shaped bowl shape having at least one of geometrically concentrated concave surface having a predetermined width (X-axis) and height (Y-axis) in a single point treatment area 465 at a focal depth (Z-axis) under the skin surface 468.

According to the eleventh embodiment, the linear ultrasonic waves may be generated using the linear ultrasound module 460, and after accurately measuring the skin thickness of the user according to the tenth embodiment, the linear ultrasound module 460 is used in consideration of the skin thickness of the user and a depth of treatment or care, and accordingly, there is a special technical effect that may exert an intensive and very optimal care effect.

### Twelfth Embodiment

FIG. 14 is a partial cross-sectional view of a facial mask according to a twelfth embodiment.

The twelfth embodiment may apply the facial masks 301 and 302 according to the above-described embodiment and the embodiments, and hereinafter, the main features of the twelfth embodiment will be mainly described.

Referring to FIG. 14, the facial mask in the twelfth embodiment may include a composite skin thickness measurement element 470.

The composite skin thickness measurement element 470 of the twelfth embodiment may include an ultrasound module 471 and a light detection module 472.

The ultrasound module 471 in the twelfth embodiment may adopt the skin thickness measurement element 450 in the tenth embodiment described above.

In addition, the light detection module 472 may include a light-emitting part 472a and a light sensor 472b. In addition, the light-emitting part 472a may be an LED, and the light sensor 472b may be a photodiode.

In the twelfth embodiment, the skin thickness may be measured by detecting a time of light reflected from the skin of the user of light emitted from the light-emitting part 472a.

According to the twelfth embodiment, the skin thickness of the user may be accurately measured by the ultrasound module 471 and the light detection module 472, and through this, the wavelength tunable light-emitting module 440 of the ninth embodiment is organically combined to control the optimal LED wavelength suitable for the corresponding user and perform skin care, and accordingly, there is a technical effect that may maximize the effect of skin care.

In addition, according to the twelfth embodiment, after accurately measuring the user's skin thickness using the linear ultrasonic module 460 of the eleventh embodiment and using the linear ultrasonic module 460 in consideration of the user's skin thickness and treatment or care depth, and accordingly, there is a special technical effect that may exert an intensive and very optimal care effect.

(Control Block Diagram of Facial Beauty Device)

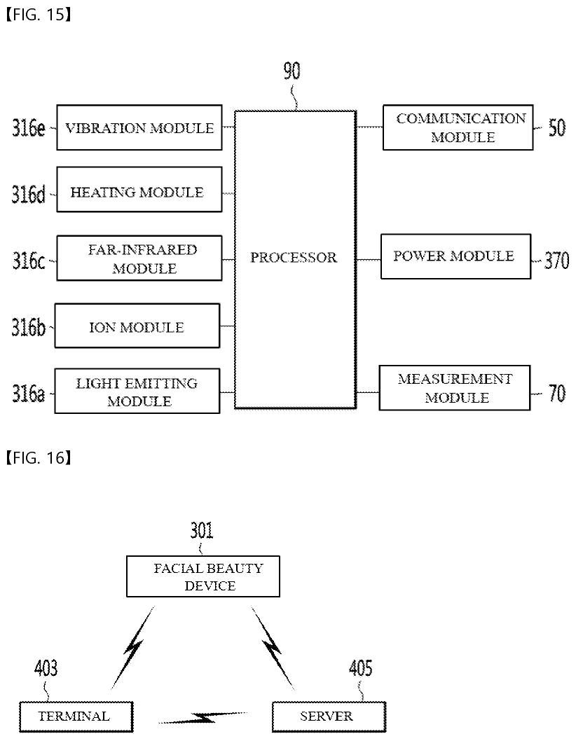

Next, FIG. 15 is a control block diagram of a facial beauty device according to an embodiment.

Referring to FIG. 15, in the embodiment, the stimulation providing part 316 of the facial beauty device 301 may include a single or a plurality of electronic devices. For example, the stimulation providing part 316 of the facial beauty device 301 may include a vibration module 316e, a heating module 316d, a far-infrared module 316c, an ion module 316b, and a light-emitting module 316a.

The far-infrared module 316c, the ion module 316b, the light-emitting module 316a, and the like may be disposed between the first face body 311 and the second face body 312. Alternatively, at least a part of the far-infrared module 316c and the ion module 316b may be disposed at the inner side of the second face body 312.

The ion module 316b may conduct electricity with the skin to supply at least one of positive and negative ions to the skin. The facial beauty device 301 may remove skin impurities by supplying positive ions to the skin and circulate blood by supplying negative ions to the skin.

The ion module 316b may supply positive ions or negative ions through an alternating current or supply positive ions or negative ions through a galvanic current. In particular, when the ion module 316b supplies positive ions or negative ions to the skin through the galvanic current, there is an advantage of supplying positive ions or negative ions to the skin while minimizing skin irritation.

In addition, the facial beauty device 301 may include at least some or all of a communication module 50, a power module 370, a measurement module 70, and a processor 90.

The processor 90 may control at least one of the vibration module 316e, the heating module 316d, the far-infrared module 316c, the ion module 316b, the light-emitting module 316a, the communication module 50, the power module 370, the measurement module 70.

The processor 90 may control so that the ion module 316b generates only positive ions in a positive ion mode, the ion module 316b generates only negative ions in a negative ion mode, and the ion module 316b generates the positive ions and negative ions alternately in a complex mode.

In addition, the ion module 316b may further generate galvanic ions.

The far-infrared module 316c may emit far-infrared light in a wavelength band having a high transmission performance among infrared rays, and through this, it is possible to transmit light energy of the far-infrared light to the inside of the skin. The far-infrared light may penetrate the skin even though it is not in direct contact with the skin, and thus there is an advantage that the light energy may be effectively transmitted.

The light-emitting module 316a may include an LED. The light-emitting module 316a may differently adjust a color emitted from the LED according to a care mode. For example, the light-emitting module 316a may emit red light in a first care mode, emit green light in a second care mode, emit blue light in a third care mode, and sequentially emit red light, green light, and blue light in a fourth care mode. As another example, the light-emitting module 316a may emit red light, green light, and blue light individually or in combination.

Meanwhile, in the present invention, the heating module 316d and the light-emitting module 316a have been described as separate configurations, but it is only described separately to distinguish functions, and thus it is reasonable that the embodiment is not limited thereto.

The stimulation providing unit may operate by receiving a control signal from a terminal 403 or a server 405 to be described later.

The measurement module 70 may include an impedance sensor for measuring moisture and oil of the skin of the user. The measurement module 70 may measure moisture, oil, and the like of the skin of the user through a measured value of the impedance sensor.

In addition, the measurement module 70 may further include an ultrasonic transducer and an ultrasonic sensor described above. The ultrasonic transducer may emit an ultrasonic beam in a form of a pulse and the ultrasonic sensor may sense that a beam emitted by the ultrasonic transducer is reflected at a boundary of the skin tissue to measure the skin thickness.

In addition, the measurement module 70 may further include a current sensor that senses an amount of current flowing through one region of second face body 312. The current sensor monitors the amount of current to determine whether the user is mounted with the facial beauty device 301. For example, the current sensor may determine that the user has mounted or removed the facial beauty device 301 when the amount of current is changed to the set value or more.

The processor 90 may set and control the vibration intensity and the number of vibrations of each of the plurality of vibration modules 316e. In addition, the processor 90 may set and control the heat generation temperature of each of the heating modules 316d.

The processor 90 may transmit and receive a signal to and from the terminal 403 or the server 405 to be described later through the communication module 50.

The processor 90 may supply power to the vibration module 316e, the heating module 316d, the communication module 50, and the processor 90 through the power module 370.

### INDUSTRIAL APPLICABILITY

(Control Block Diagram of Skin Care Device System)

FIG. 16 is a control block diagram of a skin care device system including the skin care device according to the embodiment.

The skin care device system may include the facial beauty device 301, the terminal 403, and the server 405. Each of the facial beauty device 301, the terminal 403, and the server 405 may transmit and receive a signal to and from each other. The facial beauty device 301 may transmit and receive a signal to and from the terminal 403 or the server 405 through the communication module 50.

The terminal 403 may include a smart phone, a personal computer (PC), a notebook, and the like.

Meanwhile, although only one server 405 is shown in FIG. 16, the server 405 may be implemented in plural according to a function to be described later.

According to the embodiment of the present invention, the server 405 may acquire environmental information.

The environmental information may refer to information related to the atmosphere and may include various information that may affect the human skin. For example, the environmental information may include at least one of an ultraviolet index, a temperature, a humidity, an air pollution level, an air pollution position, and the like.

The server 405 may acquire a method of operating the facial beauty device 301 based on the environmental information. The operation method may refer to operation intensity or an operation time of at least one of the vibration module 316e and the heating module 316d provided in the facial beauty device 301.

For example, the server 405 may acquire an operation method of setting the heat generating intensity of the heating module 316d to “weak” when the temperature is 30° C. or more, the heat generating intensity of the heating module 316d to “medium” when the temperature is 20° C. or more and less than 30° C., and the heat generating intensity of the heating module 316d to “strong” when the temperature is less than 20° C.

In addition, the facial beauty device 301 may have a plurality of care modes, and each of the plurality of care modes may set an operation strength or operation time of the vibration module 316e and an operation strength or operation time of the heating module 316d to be different. The server 405 may acquire the operation method of the facial beauty device 301 by determining any one of the plurality of care modes based on the environmental information.

The server 405 may transmit the operation method of the facial beauty device 301 acquired based on the environmental information to the facial beauty device 301.

Meanwhile, it has been described that a subject that acquires the operation method of the facial beauty device 301 according to the environmental information is the server 405, but it is merely illustrative. That is, after the terminal 403 acquires the environmental information, the terminal 403 may acquire the operation method of the facial beauty device 301 based on the environmental information to transmit the operation method to the facial beauty device 301, or the terminal 403 may acquire the operation method of the facial beauty device 301 based on the environmental information after the facial beauty device 301 acquires the environmental information.

According to another embodiment of the present invention, the terminal 403 may acquire the environmental information and the user information. Since the environmental information is the same as described above, duplicated descriptions will be omitted.

The user information refers to information related to the skin and skin care of the user and may include skin information of the user, a user's usage history of the skin care device, and the like. For example, the skin information may include oil content, moisture content, skin elasticity, wrinkle information, etc. of the skin of the user's face, and the usage history of the skin care device may include the number of times the facial beauty device 301 has been used, cycles, a date of recent use, preferred strength and number of times of the vibration module 316e, and preferred strength and time of the heating module 316d.

The terminal 403 may acquire the operation method of the facial beauty device 301 by considering the environmental information and the user information together and transmit the acquired operation method to the facial beauty device 301. The operation method has been described above, and thus a duplicated description will be omitted.

Meanwhile, it has been described that the subject that acquires the operation method of the facial beauty device 301 according to the environmental information and the user information is the terminal 403, but it is merely illustrative. That is, after the server 405 acquires the environmental information and the user information, the server 405 may acquire the operation method of the facial beauty device 301 to transmit the operation method to the facial beauty device 301, or the server 405 may acquire the operation method of the facial beauty device 301 after the facial beauty device 301 acquires the environmental information and the user information.

According to still another embodiment of the present invention, the facial beauty device 301 may receive an operation command from the terminal 403.

The terminal 403 may include an input unit (not shown) for receiving an operation input from the user, and the terminal 403 may transmit the operation command to the facial beauty device 301 so that the facial beauty device 301 operates according to the operation input received from the input unit (not shown).

As an example, the terminal 403 may display a plurality of care modes and receive an input for selecting any one of the plurality of care modes through the input unit (not shown).

The characteristics, structures, effects, and the like described in the above-described embodiments are included in at least one embodiment of the present invention, but are not limited to only one embodiment. Furthermore, the characteristic, structure, and effect illustrated in each embodiment may be combined or modified for other embodiments by a person skilled in the art. Thus, it should be construed that the contents related to such combination and modification are included in the scope of the present invention.

In addition, the above description has been focused on the embodiments, but it is merely illustrative and does not limit the present invention. Those skilled in the art to which the embodiments pertain may appreciate that various modifications and applications not illustrated above are possible without departing from the essential features of the embodiment. For example, each component particularly represented in the embodiments may be modified and realized. In addition, it should be construed that differences related to such a modification and an application are included in the scope of the present invention defined in the appended claims.

1. A facial beauty device comprising:
a face body;
a mounting member coupled to the face body; and
a support part disposed at one side of the face body,
wherein the face body includes
a first face body, a second face body, and a circuit board disposed between the first face body and the second face body,
the circuit board includes a predetermined stimulation providing part disposed thereon, and
the second face body includes a mask sheet disposed at an inner side thereof.

2. The facial beauty device of claim 1, wherein the mask sheet includes a predetermined opening so that an electrode provided in the stimulation providing part can be energized to a user's face even after the mask sheet is attached.

3. The facial beauty device of claim 2, wherein the mask sheet includes a plurality of openings,
the mask sheet includes a first opening in an area corresponding to the user's eyes, a second opening in an area corresponding to the user's mouth, and a third opening at a position corresponding to the stimulation providing part.

4. The facial beauty device of claim 3, wherein a second diameter of the third opening of the mask sheet is larger than a first diameter of the stimulation providing part.

5. The facial beauty device of claim 1, wherein a hardness of the first face body and that of the second face body are different from each other.

6. The facial beauty device of claim 1, wherein the stimulation providing part includes one or more among an ion module, a vibration module, and a light-emitting module.

7. The facial beauty device of claim 1, wherein the mounting member includes a first mounting member coupled to a first region of the second face body and a second mounting member coupled to a second region of the second face body.

8. The facial beauty device of claim 1, further comprising
a first frame on the second face body,
wherein the first frame includes a first-first frame disposed at a position corresponding around the user's eye area and
a first-third frame disposed at a position corresponding around the user's forehead.

9. The facial beauty device of claim 8, wherein the first frame includes a first-second frame disposed between the first-first frame and the first-third frame.

10. The facial beauty device of claim 9, wherein a horizontal width of the first-first frame is smaller than a vertical width of the first-second frame.

11. A facial beauty device comprising:
a face body;
a mounting member coupled to the face body; and
a support part disposed on one side of the face body,
wherein the face body includes a first face body, a second face body, and a circuit board disposed between the first face body and the second face body, and
the circuit board includes a predetermined stimulation providing part thereon.

12. The facial beauty device of claim 1, wherein the stimulation providing part includes a first light-emitting module disposed on the face body and
further includes a first diffusion plate disposed on the first light-emitting module.

13. The facial beauty device of claim 1, wherein the stimulation providing part includes a second light-emitting module disposed on the circuit board and
further includes a second diffusion plate disposed on a side surface of the second light-emitting module and a reflective material coating layer disposed above the second diffusion plate.

14. The facial beauty device of claim 1, wherein the stimulation providing part includes a first light-emitting module disposed on the circuit board and
further includes a first diffusion plate disposed on the first light-emitting module and a diffraction optical pattern disposed between the first diffusion plates.

15. The facial beauty device of claim 1, wherein the stimulation providing part includes a piezoelectric sensor disposed on the circuit board, and
the piezoelectric sensor includes
a flexible substrate, a piezoelectric thin film layer on the flexible substrate, an electrode on the piezoelectric thin film layer, and a protective layer on the electrode.

16. The facial beauty device of claim 11, further comprising
a mask sheet disposed at an inner side of the second face body and
an adhesive layer at an edge of the inner side of the second face body.

17. The facial beauty device of claim 11, further comprising a mask sheet disposed at an inner side of the second face body,
wherein the stimulation providing part includes a contact time sensing element disposed on the circuit board,
wherein the contact time sensing element includes a plurality of electrical contacts and a plurality of ports, and
detects a short-circuit between the electrical contacts by a monitor module to determine whether a formulation of the mask sheet remains.

18. The facial beauty device of claim 1, wherein the stimulation providing part includes an optical vibration composite element disposed on the circuit board,
wherein the optical vibration composite element includes
a first electrode layer disposed on the circuit board, a flexible light-emitting layer disposed on the first electrode layer, a second electrode layer disposed on the flexible light-emitting layer, a flexible ultrasound layer disposed on the second electrode layer, and a third electrode layer disposed on the flexible ultrasound layer.

19. The facial beauty device of claim 1, wherein at least one of an optical vibration composite element, a wavelength tunable light-emitting module, a skin thickness measurement element, a linear ultrasound module, and a composite skin thickness measurement element is disposed on the circuit board.

20. A mask sheet used in a facial beauty device having a stimulation providing part on the face body of claim 1, the mask sheet comprising
an opening through which a predetermined electrode provided in the stimulation providing part of the facial beauty device can pass by providing a predetermined opening.
